(19) United States
(12) Patent Application Publication
(10) Pub. No.: US 2022/0299528 A1
(43) Pub. Date: Sep. 22, 2022

(54) REAGENT, TEST PAPER, REAGENT KIT, AND TEST PAPER KIT FOR DETECTING SULFHYDRYL COMPOUND, AND PREPARATION METHOD THEREOF

(71) Applicant: HANGZHOU AOGO MEDICAL CO., LTD., Hangzhou (CN)

(72) Inventors: Min LUO, Hangzhou (CN); Zhijian HUANG, Hangzhou (CN); Junfeng ZHANG, Hangzhou (CN)

(73) Assignee: HANGZHOU AOGO MEDICAL CO., LTD., Hangzhou (CN)

(21) Appl. No.: 17/636,837

(22) PCT Filed: Aug. 19, 2020
(86) PCT No.: PCT/CN2020/109906

§ 371 Date: Feb. 18, 2022

(30) Foreign Application Priority Data
Aug. 19, 2019 (CN) 2019 10762052.5

(51) Int. Cl. G01N 33/68; G01N 33/52

(52) U.S. Cl. CPC G01N 33/6893; G01N 33/523; G01N 2800/26

(57) ABSTRACT

A reagent, test paper, a reagent kit, and a test paper kit for detecting a sulfhydryl compound, and a preparation method thereof are provided. The reagent is obtained by mixing a phosphotungstic acid (PTA) reagent, an acetate buffer, and a shield reagent in a specified ratio. The test paper is composed of porous water-absorbent paper and a dry detection reagent dispersed thereon. The reagent is based on principle innovation. When in use, the reagent is directly mixed with urine to be tested in a volume ratio of 2:1, a resulting mixture is allowed to stand for 10 min to 15 min, and then a result can be directly determined by naked eyes. The reagent overcomes the shortcoming of requiring a control sample to assist in the determination of a result in existing methods, and has the advantages of simple operation, safety and non-toxicity, and rapid detection.

### CROSS REFERENCE TO THE RELATED APPLICATIONS

This application is the national phase entry of International Application No. PCT/CN2020/109906, filed on Aug. 19, 2020, which is based upon and claims priority to Chinese Patent Application No. 201910762052.5, filed on Aug. 19, 2019, the entire contents of which are incorporated herein by reference.

### TECHNICAL FIELD

The present disclosure relates to the technical field of detection reagents and test papers, and in particular to a reagent, test paper, a reagent kit, and a test paper kit for detecting a sulfhydryl compound, and a preparation method thereof.

### BACKGROUND

Human papillomavirus (HPV) belongs to the family Papillomaviridae and is a small-molecule, non-enveloped circular double-stranded DNA virus. HPV infects humans through a direct or indirect contact with contaminated items or through sexual transmission. The virus is both host-specific and tissue-specific, and can only infect human skin and mucosal epithelial cells, causing various papillomas or warts of human skin and epithelial proliferative damage to the reproductive tract. More than 200 HPV subtypes have been found, about 40 of which can infect the reproductive tract. According to pathogenicity, the HPV subtypes are divided into a high-risk type and a low-risk type, which show regional, ethnic, and age differences in infection.

(1) There are some regional differences in the distribution of HPV subtypes worldwide, and different HPV subtypes lead to different infections and consequences. Subtype 16 is the main HPV subtype worldwide, followed by subtypes 18, 33, 52, and 58. Subtypes 16, 18, and 33 are the main subtypes in Asia. Subtypes 16, 18, 52, and 58 are most common in China. Common subtypes in cervical precancerous lesions are HPV 16, 58, 52, 18, 33, and 31.

(2) The high-risk types include HPV 16, 18, 31, 33, 35, 39, 45, 54, 52, 53, 56, 58, 59, 66, 58, 73, and 82, which mainly cause external genital cancer, cervical cancer, high-grade vulvar and cervical intraepithelial neoplasia, and malignant lesions elsewhere.

(3) The low-risk types include HPV 6, 11, and 42, which mainly induce external genital and skin condyloma acuminatum, low-grade vulvar and cervical intraepithelial neoplasia, and warty lesions and low-grade intraepithelial neoplasia elsewhere.

(4) The low-risk type can usually be detected in benign or low-grade cervical lesions, while the high-risk type is usually found in high-grade lesions and cervical cancer. Patients with HPV infection generally have no clinical discomfort, and a few have symptoms such as genital itching, genital burning pain, and increased leucorrhea. After HPV infection, the labia majora, labia minora, clitoris, vagina, cervix, or perianal area has small reddish or gray papules that are verrucous protrusions arranged in clusters or fused into cockscombs or cauliflower-like vegetations, increasing fragility of skin lesions to cause bleeding. The persistent infection of high-risk HPV is the main cause of cervical cancer and precancerous lesions thereof. The detection of HPV infection has become an important basis for the prevention, diagnosis, and treatment of cervical lesions.

(5) Women are more susceptible to high-risk HPV than men, while men are more susceptible to low-risk HPV than women. HPV infection in males is related to HPV infection in female sexual partners thereof to some extent. If a person has an infected sexual partner, has multiple sexual partners in recent 3 to 6 months or a sexual partner who has multiple sexual partners, and has gonorrhea, syphilis, chlamydia infection, trichomonal vaginitis, and the like, suspected HPV infection can be self-diagnosed. For suspected HPV infection, a gauze soaked in a 3% to 5% acetic acid solution is wrapped around or applied to a suspicious skin or mucous membrane surface, and removed 3 min to 5 min later. Typical condyloma acuminatum lesions will be manifested as white papules or warts, and subclinical infection will be manifested as white patches or spots. An acetowhite test is a simple and easy detection method to identify early condyloma acuminatum lesions and subclinical infection. If HPV or suspected infection is found, a person should go to the hospital for confirmed diagnosis.

(6) Cervical precancerous lesions may occur only after 8 to 24 months of persistent HPV infection, and there is no morphological change before, which is a blind spot of morphological analysis. It is impossible to find abnormal cell morphology through histopathology and cytology, as only a negative result can be reported. However, since changes in gene mutation and sulfhydryl shedding occur in the process at a molecular level, determination of sulfhydryl in urine can be conducted to yield a positive result. False negative rate of diagnosis of cervical cancer and cervical precancerous lesions. The occurrence and development of cervical cancer is a gradual evolution process, and the excretion of sulfhydryl varies at different stages of a lesion. On the whole, more and more sulfhydryl groups are excreted from the early stage to advanced stage of a lesion.

Existing sulfhydryl compound detection methods are all designed for the reducibility of sulfhydryl compounds. By oxidizing a sulfhydryl compound, an oxidizing reagent is reduced to show a color, and then a sulfhydryl compound content is determined by colorimetry, or whether a result is positive is judged by directly observing the color. However, since urine or secretions also include other reducing substances, the current detection methods require the addition of a control sample to determine a result. Generally, mercuric chloride is added to shield a sulfhydryl compound such that the sulfhydryl compound does not react with an oxidizing reagent, thereby decreasing a degree of color development as a control. However, mercuric chloride is a highly-toxic reagent, which can cause operational hazards and environmental pollution. In addition, when a control sample is adopted for detection, there should be a significant difference in color development between the control sample and a test sample, otherwise it is difficult to determine a result only by naked eyes.

### SUMMARY

In order to solve the problems in the prior art, a first objective of the present disclosure is to provide a sulfhydryl compound detection reagent.

A second objective of the present disclosure is to provide a preparation method of the sulfhydryl compound detection reagent.

A third objective of the present disclosure is to provide a reagent kit including the sulfhydryl compound detection reagent described above.

A fourth objective of the present disclosure is to provide urine sulfhydryl compound detection test paper, a preparation method of the detection test paper, and a test paper kit.

In order to solve the technical problems, the present disclosure adopts the following technical solutions:

The sulfhydryl compound detection reagent provided by the present disclosure is prepared by mixing three reagent components, including a phosphotungstic acid (PTA) reagent, an acetate buffer, and a shield reagent; and the shield reagent is oxidative, transparent, and colorless, and remains colorless after being reduced under specified conditions.

A working principle of the present disclosure is as follows: When a detection object is normal human urine or a secretion (which also includes a reducing substance such as vitamin C), a general PTA reagent will rapidly develop a color and turn blue when reacting with vitamin C, resulting in a false positive result; and after the shield reagent is added, the vitamin C preferentially reacts with the shield reagent and will not react with the PTA reagent to cause color development, resulting in a normal negative result. When a detection object is a patient with HPV infection, a large amount of sulfhydryl compound is included (a sulfhydryl compound content is much higher than a reducing substance content in normal urine or secretions, in which case the shield reagent preferentially reacts with the sulfhydryl compound, but is not enough to completely shield the sulfhydryl compound. Moreover, highly-active reducing substances in normal urine or secretions are protected, and react with the PTA reagent to cause color development, resulting in a true positive result, that is, the original highly-active reducing substances in urine or secretions serve as indication reactants for the sulfhydryl compound.

In addition, uric acid of a high content in human urine has reducibility and shows high reducibility under alkaline conditions, but the detection reagent provided by the present disclosure is a slightly-acidic reagent. Therefore, the interference of uric acid can be excluded. If other secretions include uric acid reducing substances, there is usually high reducibility under alkaline conditions, and similarly, due to the acidic conditions of the detection reagent, the interference can be excluded.

Preferably, a preparation method of the PTA reagent may be as follows: weighing 5±0.5 g of sodium tungstate, dissolving the sodium tungstate in 40±5 ml of deionized water, and adding 4±0.5 ml of 85±0.5% concentrated phosphoric acid and zeolite; heating a resulting mixture to reflux, starting timing, and continuously heating under reflux for 2±0.5 h; and stopping heating, cooling to room temperature, diluting to 100±0.5 ml, and storing a resulting product in a brown reagent bottle.

Preferably, a preparation method of the acetate buffer may be as follows: preparing a 2±0.5 M sodium acetate solution and a 2±0.5 M acetic acid solution separately, and mixing the two solutions in a volume ratio of 5:1.

Preferably, a preparation method of the shield reagent may be as follows: weighing 25 mg to 100 mg of a shield agent, dissolving the shield agent in 100±0.5 ml of deionized water, and storing a resulting product in a brown reagent bottle.

Preferably, the sulfhydryl compound detection reagent may be obtained by thoroughly mixing the PTA reagent, the acetate buffer, and the shield reagent in a volume ratio of 2:1:1.

A preparation method of a sulfhydryl compound detection reagent is provided, including the following steps:

S1: weighing 5±0.5 g of sodium tungstate, dissolving the sodium tungstate in 40±5 ml of deionized water, and adding 4±0.5 ml of 85±0.5% concentrated phosphoric acid and zeolite; heating a resulting mixture to reflux, starting timing, and continuously heating under reflux for 2±0.5 h; and stopping heating, cooling to room temperature, diluting to 100±0.5 ml, and storing a resulting PTA reagent in a brown reagent bottle;

S2: preparing a 2±0.5 M sodium acetate solution and a 2±0.5 M acetic acid solution separately, and mixing the two solutions in a volume ratio of 5:1 to obtain an acetate buffer;

S3: weighing 50 mg to 100 mg of a shield agent, dissolving the shield agent in 100±0.5 ml of deionized water, and storing a resulting shield reagent in a brown reagent bottle; and

S4: thoroughly mixing the PTA reagent, the acetate buffer, and the shield reagent in a volume ratio of 2:1:1.

The present disclosure also provides a sulfhydryl compound self-examination kit, mainly including a sampler, a test reagent, and a test tube: and the test reagent is the aforementioned sulfhydryl compound detection reagent of the present disclosure.

The present disclosure provides urine sulfhydryl compound detection test paper composed of a porous water-absorbent paper and a dry detection reagent dispersed thereon, where the detection reagent is prepared by mixing three reagents, including a PTA reagent, an acetate buffer, and a shield reagent; and the shield reagent is oxidative, transparent, and colorless, and remains colorless after being reduced under specified conditions.

A preparation method of the urine sulfhydryl compound detection test paper of the present disclosure is as follows: after the detection reagent is prepared, immersing porous water-absorbent paper in the detection reagent; and after the porous water-absorbent paper fully absorbs the detection reagent, taking out the porous water-absorbent paper, and draining and drying the porous water-absorbent paper to obtain the sulfhydryl compound detection test paper.

The present disclosure also provides a urine sulfhydryl compound self-examination kit, mainly including: a urine sampler, the detection test paper of the present disclosure, and a desiccant.

The present disclosure is based on the following principle: When the test paper contacts urine, if the urine is normal urine, vitamin C in the urine preferentially reacts with the shield reagent and does not react with the PTA reagent, such that the test paper does not develop a color, indicating a negative result; and if the urine includes a large amount of sulfhydryl compound, the sulfhydryl compound in the urine preferentially reacts with the shield reagent, and then vitamin C reacts with the PTA reagent, such that the test paper turns blue, indicating a positive result, in which case vitamin C serves as an indicator of the sulfhydryl compound.

Preferably, a preparation method of the PTA reagent may be as follows: weighing 5±0.5 g of sodium tungstate, dissolving the sodium tungstate in 40±5 ml of deionized water, and adding 4±0.5 ml of 85±0.5% concentrated phosphoric acid and zeolite; heating a resulting mixture to reflux, starting timing, and continuously heating under reflux for 2±0.5 h; and stopping heating, cooling to room temperature, diluting to 100±0.5 ml, and storing a resulting product in a brown reagent bottle.

Preferably, a preparation method of the acetate buffer may be as follows: preparing a 2±0.5 M sodium acetate solution and a 2±0.5 M acetic acid solution separately, and mixing the two solutions in a volume ratio of 5:1.

Preferably, a preparation method of the shield reagent may be as follows: weighing 25 mg to 100 mg of a shield agent, dissolving the shield agent in 100±0.5 ml of deionized water, and storing a resulting product in a brown reagent bottle.

Preferably, the shield agent may be sodium iodate and/or sodium periodate.

Preferably, the sulfhydryl compound detection reagent may be obtained by thoroughly mixing the PTA reagent, the acetate buffer, and the shield reagent in a volume ratio of 2:1:0.5.

A preparation method of urine sulfhydryl compound detection test paper is provided, including the following steps:

step 1: preparation of a detection reagent

S1: weighing 5±0.5 g of sodium tungstate, dissolving the sodium tungstate in 40±5 ml of deionized water, and adding 4±0.5 ml of 85±0.5% concentrated phosphoric acid and zeolite; heating a resulting mixture to reflux, starting timing, and continuously heating under reflux for 2±0.5 h; and stopping heating, cooling to room temperature, diluting to 100±0.5 ml, and storing a resulting PTA reagent in a brown reagent bottle;

S2: preparing a 2±0.5 M sodium acetate solution and a 2±0.5 M acetic acid solution separately, and mixing the two solutions in a volume ratio of 5:1 to obtain an acetate buffer;

S3: weighing 50 mg to 100 mg of a shield agent, dissolving the shield agent in 100±0.5 ml of deionized water, and storing a resulting shield reagent in a brown reagent bottle; and

S4: thoroughly mixing the PTA reagent, the acetate buffer, and the shield reagent in a volume ratio of 2:1:0.5;

step 2: immersing porous water-absorbent paper in the detection reagent; and after the porous water-absorbent paper fully absorbs the detection reagent, taking out the porous water-absorbent paper, and draining and drying the porous water-absorbent paper to obtain the sulfhydryl compound detection test paper.

Preferably, the porous water-absorbent paper may be filter paper; the porous water-absorbent paper may be immersed at room temperature for no less than 5 seconds; and the porous water-absorbent paper is vacuum-dried at 40° C. to 60° C.

More preferably, when the porous water-absorbent paper is immersed, an ultrasonic-assisted treatment may be conducted for no less than 10 seconds. The ultrasonic-assisted treatment can make the detection reagent fully enter and store in three-dimensional (3D) micropores of the detection test paper, which avoids the problem that local voids can be loaded with limited detection reagent, improves the uniformity of distribution of the detection reagent on the detection test paper, and improves the detection reliability of the detection test paper.

The present disclosure has the following beneficial effects:

The sulfhydryl compound detection reagent provided by the present disclosure is based on principle innovation, and has the advantages of safety and non-toxicity, simple operation, rapid detection, no need for control, and the like.

The urine sulfhydryl compound detection test paper provided by the present disclosure has the advantages of stability, long shelf life, safety and non-toxicity, simple operation, rapid detection, no need for control, and the like.

Given that sulfhydryl components in urine are complex and HPV infection does not necessarily cause gene mutation of cervical cancer and cervical precancerous lesions, the detection of sulfhydryl in urine to determine whether a patient is infected with or has been infected with HPV also has some limitations, and may lead to false positive and false negative.

False positive:

1. Theoretically, from a qualitative concept, sulfhydryl will be shed when gene mutations occur in various histiocytes. Data of clinical trials conducted by Cancer Hospital of Chinese Academy of Medical Sciences on some common cancers in China (gastric cancer, lung cancer, liver cancer, breast cancer, colon cancer, nasopharyngeal carcinoma (NPC), esophageal cancer, and the like) show that, in the use of a urine sulfhydryl detection method according to specified standards, a positive rate of malignant tumors other than cervical cancer (with a positive rate of 80%/6 or higher) is 12%.

2. Pathogenic microorganisms of vaginitis include Gardnerella vaginalis (G. vaginalis), Trichomonas vaginalis (T. vaginalis), Candida albicans (C. albicans), and the like, and it cannot be excluded that these microorganisms will produce sulfhydryl metabolites during infection, resulting in a positive result for a urine sample.

3. Other reducing substances (such as vitamin C and uric acid), some trace components and abnormal metabolites caused by other etiological factors in urine or secretions may lead to false positive results.

4. Improper operations may lead to false positive results.

5. For some patients with sulfhydryl in urine, a current HPV nucleic acid test result may not be positive, but these patients may have a history of HPV infection and thus may at a gene mutation stage before precancerous lesion. Therefore, for these patients with false positive results, the screening of populations at a high risk of cervical precancerous lesions or cervical cancer is more significant than the detection of a current positive HPV infection.

False negative:

1. There are more than 130 types of HPV that have been isolated. The chance of women being infected with HPV in their lifetime is as high as 80%, but HPV in a large number of infected patients will be automatically removed by the body's immune system, which results in no gene mutation in histiocytes and does not cause the continuous increase of sulfhydryl in urine. Therefore, a false negative rate will be high for people with low-risk transient infection and people whose early HPV infection has not caused gene mutation in cell tissues. However, a false negative rate will be greatly reduced for people with high-risk persistent HPV infection screened by detecting sulfhydryl in urine, as such, people with high-risk persistent HPV infection are of the greatest significance to be screened.

2. Tumors, stones, inflammation, and the like cause blockage of the urinary tract, such that shed sulfhydryl cannot be smoothly excreted through the urinary tract.

3. When a patient is not deprived of water and food or the collected urine is not the first morning urine, a relative concentration of sulfhydryl in the urine is low.

4. People with color blindness and color weakness cannot observe a reaction color correctly, resulting in the wrong determination of a reaction result.

5. Factors such as irregular detection operations will also cause some man-induced false negative results.

### DETAILED DESCRIPTION OF THE EMBODIMENTS

Based on the principles of the present disclosure, the technical solutions of the present disclosure are further described below through specific examples. All reagents used in the examples are purchased from the market, and are analytically pure.

Specific preparation steps of the sulfhydryl compound detection reagent provided by the present disclosure are as follows:

S1: preparation of a PTA reagent: weighing 5 g of sodium tungstate, dissolving the sodium tungstate in 40 ml of deionized water, and adding 4 ml of 85% concentrated phosphoric acid and zeolite; heating a resulting mixture to reflux, starting timing, and continuously heating under reflux for 2 h; and stopping heating, cooling to room temperature, diluting to 100 ml, and storing a resulting PTA reagent in a brown reagent bottle;

S2: preparation of an acetate buffer: preparing a 2 M sodium acetate solution and a 2 M acetic acid solution separately, and mixing the two solutions in a volume ratio of 5:1 to obtain the acetate buffer;

S3: preparation of a shield reagent: weighing 25 mg to 100 mg (preferably 50 mg to 100 mg) of a shield agent, dissolving the shield agent in 100 ml of deionized water, and storing a resulting shield reagent in a brown reagent bottle; and

S4: preparation of the sulfhydryl compound detection reagent: thoroughly mixing the PTA reagent, the acetate buffer, and the shield reagent in a volume ratio of 2:1:1.

The present disclosure is used for detection as follows: urine and a sulfhydryl compound detection reagent are mixed in a volume ratio of 1:2 and a resulting mixture is allowed to stand for 10 min to 15 min, where if the mixture turns blue obviously, it indicates a positive result; otherwise it indicates a negative result.

As described in S3, the shield agent is an oxidant soluble in water; when the shield agent is dissolved, a resulting solution is transparent and colorless, and remains colorless after the shield agent is reduced under specified conditions; and preferably the shield agent is sodium iodate or sodium periodate.

In order to make the present disclosure clearer and more comprehensible, the present disclosure will be further described in detail below with reference to the examples. It should be understood that the examples described herein are merely intended to explain the present disclosure, rather than to limit the present disclosure.

### Example 1

### Preparation of a Sulfhydryl Compound Detection Reagent

preparation of a PTA reagent: 5 g of sodium tungstate was weighed and dissolved in 40 ml of deionized water, and 4 ml of 85% concentrated phosphoric acid and zeolite were added; a resulting mixture was heated to reflux, timing was started, and the mixture was continuously heated under reflux for 2 h; and the heating was stopped, and a resulting product was cooled to room temperature, diluted to 100 ml, and stored in a brown reagent bottle;

preparation of an acetate buffer: a 2 M sodium acetate solution and a 2 M acetic acid solution were prepared separately, and mixed in a volume ratio of 5:1;

preparation of a shield reagent: 50 mg of sodium iodate was weighed and dissolved in 100 ml of deionized water, and a resulting product was stored in a brown reagent bottle; and

preparation of the sulfhydryl compound detection reagent: the PTA reagent, the acetate buffer, and the shield reagent were thoroughly mixed in a volume ratio of 2:1:1.

### Example 2

### Preparation of a Sulfhydryl Compound Detection Reagent

preparation of a shield reagent: 100 mg of sodium iodate was weighed and dissolved in 100 ml of deionized water, and a resulting product was stored in a brown reagent bottle; and the rest of the steps were the same as those in Example 1.

### Test Example 1

The effect of the detection reagent of the present disclosure was tested on simulated urine samples below.

Preparation of simulated urine sample 1: 500 mg of L-cysteine and 7 mg of vitamin C were weighed and dissolved in 100 ml of water.

Preparation of simulated urine sample 2: 7 mg of vitamin C was weighed and dissolved in 100 ml of water.

100 μl of the simulated urine sample 1 was taken and mixed with 200 μl of the detection reagent in Example 1, a resulting mixture was allowed to stand for 10 min to 15 min, and then the mixture turned blue, indicating a positive result. 100 μl of the simulated urine sample 2 was taken and mixed with 200 μl of the detection reagent in Example 1, a resulting mixture was allowed to stand for 10 min to 15 min, and the color of the mixture did not change, indicating a negative result.

### Test Example 2

The effect of the detection reagent of the present disclosure was tested on normal human urine samples below.

Normal human urine sample 1: urine excreted for the first time after breakfast was collected and cooled to room temperature by standing.

Normal human urine sample 2: 25 ml of the normal human urine sample 1 was taken, and 125 mg of L-cysteine was added and dissolved.

Preparation of a PTA reagent without a shield reagent: the PTA reagent in Example 1 and the acetate buffer were thoroughly mixed in a volume ratio of 2:1.

100 μl of the normal human urine sample 1 was taken and mixed with 200 μl of the detection reagent in Example 2, a resulting mixture was allowed to stand for 10 min to 15 min, and a color of the mixture did not change, indicating a negative result; 100 μl of the simulated urine sample 2 was taken and mixed with 200 μl of the detection reagent in Example 2, a resulting mixture was allowed to stand for 10 min to 15 min, and then a color of the mixture turned blue, indicating a positive result; and 100 μl of the normal human urine sample 1 was taken and mixed with 200 μl of the PTA reagent without a shield reagent, a resulting mixture was allowed to stand for 10 min to 15 min, and then a color of the mixture turned blue, indicating a false positive result.

The preparation of the sulfhydryl compound detection test paper of the present disclosure is described below with reference to some examples:

Specific preparation steps of the sulfhydryl compound detection test paper provided by the present disclosure were as follows:

S1: preparation of a PTA reagent: 5 g of sodium tungstate was weighed and dissolved in 40 ml of deionized water, and 4 ml of 85% concentrated phosphoric acid and zeolite were added; a resulting mixture was heated to reflux, timing was started, and the mixture was continuously heated under reflux for 2 h; and heating was stopped, a resulting product was cooled to room temperature, diluted to 100 ml, and stored in a brown reagent bottle;

S2: preparation of an acetate buffer: a 2 M sodium acetate solution and a 2 M acetic acid solution were prepared separately and mixed in a volume ratio of 5:1;

S3: preparation of a shield reagent: 25 mg to 100 mg (preferably 50 mg to 100 mg) of a shield agent was weighed and dissolved in 100 ml of deionized water, and a resulting product was stored in a brown reagent bottle;

S4: preparation of the sulfhydryl compound detection reagent: the PTA reagent, the acetate buffer, and the shield reagent were thoroughly mixed in a volume ratio of 2:1:0.5; and

filter paper was immersed in the prepared urine sulfhydryl compound detection reagent, and after the filter paper fully absorbed the detection reagent, the filter paper was taken out, drained, and dried at 40° C. under vacuum conditions.

Urine test: a specified amount of urine was taken and added dropwise on the test paper, and then the test paper stood for air-drying and observed, wherein if a color of the test paper turned blue, it indicated a positive result, otherwise it indicated a negative result.

In order to make the present disclosure clearer and more comprehensible, the present disclosure will be further described in detail below with reference to the examples. It should be understood that the examples described herein are merely intended to explain the present disclosure, rather than to limit the present disclosure.

### Example 3

### Preparation of Sulfhydryl Compound Detection Test Paper

preparation of a PTA reagent: 5 g of sodium tungstate was weighed and dissolved in 40 ml of deionized water, and 4 ml of 85% concentrated phosphoric acid and zeolite were added; a resulting mixture was heated to reflux, timing was started, and the mixture was continuously heated under reflux for 2 h; and the heating was stopped, and a resulting product was cooled to room temperature, diluted to 100 ml, and stored in a brown reagent bottle;

preparation of an acetate buffer: a 2 M sodium acetate solution and a 2 M acetic acid solution were prepared separately, and mixed in a volume ratio of 5:1;

preparation of a shield reagent: 50 mg of sodium iodate was weighed and dissolved in 100 ml of deionized water, and a resulting product was stored in a brown reagent bottle;

preparation of the sulfhydryl compound detection reagent: the PTA reagent, the acetate buffer, and the shield reagent were thoroughly mixed in a volume ratio of 2:1:0.5; and

filter paper was immersed in the prepared urine sulfhydryl compound detection reagent, an ultrasonic-assisted treatment was conducted for 10 s, and after the filter paper fully absorbed the detection reagent, the filter paper was taken out, drained, and dried at 40° C. under vacuum conditions.

### Example 4

### Preparation of Sulfhydryl Compound Detection Test Paper

preparation of a shield reagent: 100 mg of sodium iodate was weighed and dissolved in 100 ml of deionized water, and a resulting product was stored in a brown reagent bottle;

filter paper was immersed in the prepared urine sulfhydryl compound detection reagent, and after the filter paper fully absorbed the detection reagent, the filter paper was taken out, drained, and dried at 60° C. under vacuum conditions; and

the rest of the steps were the same as those in Example 3.

### Test Example 3

The effect of the detection reagent of the present disclosure was tested on simulated urine samples below.

Preparation of simulated urine sample 1: 500 mg of L-cysteine and 7 mg of vitamin C were weighed and dissolved in 100 ml of water.

Preparation of simulated urine sample 2: 7 mg of vitamin C was weighed and dissolved in 100 ml of water.

The simulated urine sample 1 was taken and added dropwise on the test paper prepared in Example 3, the test paper stood for air-drying, and then the test paper turned blue, indicating a positive result; and the simulated urine sample 2 was taken and added dropwise on the test paper prepared in Example 3, the test paper stood for air-drying, and then a color of the test paper did not change, indicating a negative result.

### Test Example 4

The effect of the detection reagent of the present disclosure was tested on normal human urine samples below.

Normal human urine sample 1; urine excreted for the first time after breakfast was collected and cooled to room temperature by standing.

Normal human urine sample 2: 25 ml of the normal human urine sample 1 was taken, and 125 mg of L-cysteine was added and dissolved.

Preparation of a PTA reagent without a shield reagent: the PTA reagent in Example 1 and the acetate buffer were thoroughly mixed in a volume ratio of 2:1. Then filter paper was immersed in the reagent, after the filter paper fully absorbed the reagent, the filter paper was taken out, drained, and dried at 60° C.

The normal human urine sample 1 was taken and added dropwise on the test paper prepared in Example 4, the test paper stood for air-drying, and then the test paper turned slightly yellow, indicating a negative result, the normal human urine sample 2 was taken and added dropwise on the test paper prepared in Example 4, the test paper stood for air-drying, and then the test paper turned blue, indicating a positive result; and the normal human urine sample 1 was taken and added dropwise on the PTA test paper without a shield reagent, the test paper stood for air-drying, and then the test paper turned blue, indicating a false positive result.

Based on the reagent, test paper, reagent kit, and test paper kit for detecting a sulfhydryl compound of the present disclosure, when a positive test result is preliminarily obtained, in order to allow a self-diagnosed patient to further understand an inducement of their HPV infection before seeking medical treatment and facilitate clinicians to understand clinical symptoms and signs, the present disclosure also designs the following self-diagnosis outline and treatment and prevention measures for the reference of self-diagnosed patients and physicians:

I. Incentive: Do you have any of the following conditions before feeling discomfort?

1. Your spouse has been infected.

2. In the past 3 to 6 months, you have had multiple sexual partners or your sexual partner has had multiple sexual partners.

3. Whether you have recently suffered from gonorrhea, syphilis, chlamydia infection, trichomonal vaginitis, and the like.

4. Do you wear short shorts and go out to public places in summer and do you wash your hands before going to the toilet?

5. Do you use public bathtubs, bedpans, bath towels, and bathrobes?

II. Clinical symptoms: Where do you feel uncomfortable?

1. You are not uncomfortable.

2. You feel genital itching.

3. Your leucorrhea increases.

4. You feel genital burning pain.

III. Clinical signs: What did you see or touch?

1. Growth parts: labia majora, labia minora, clitoris, vagina, cervix, or perianal area, two of which commonly have papules at the same time.

2. Local manifestations: small reddish or gray papules that are verrucous protrusions arranged in clusters or fused into cockscombs, cauliflower-like vegetations, and increased fragility of skin lesions causing bleeding.

IV. Laboratory examination: Self-test

1. A urine sulfhydryl test is conducted, and a positive result is obtained.

2. An acetowhite test is conducted as follows: a gauze soaked in a 3% to 5% acetic acid solution is wrapped around or applied to a suspicious skin or mucous membrane surface, and removed 3 min to 5 min later. Typical condyloma acuminatum lesions will be manifested as white papules or warts, and subclinical infection will be manifested as white patches or spots.

The acetowhite test is a simple and easy detection method to identify early condyloma acuminatum lesions and subclinical infection.

V: Treatment

1. If HPV infection is discovered, you go to the hospital for confirmed diagnosis.

2. If HPV infection is suspected, you go to the hospital for confirmed diagnosis.

VI: Prevention

1. Sexual partners are fixed to each other and pay attention to sexual hygiene.

2. Condoms can reduce the probability of obvious HPV infection.

3. In summer, you should wear skirts or pants that are long enough to reach knee joints before going to public places.

4. You should wash your hands thoroughly before going to the toilet, and do not directly contact the toilet door with your hands (20% of people infected with HPV may carry the virus on their fingers).

The above examples are merely preferred solutions of the present disclosure and are not intended to limit the present disclosure in any form, and other variations and modifications may be made without departing from the technical solutions of the present disclosure as set forth in the appended claims.

What is claimed is:

1. A sulfhydryl compound detection reagent, wherein the sulfhydryl compound detection reagent is composed of a phosphotungstic acid (PTA) reagent, an acetate buffer, and a shield reagent; and the shield reagent is oxidative, transparent, and colorless, and the shield reagent remains colorless after being reduced under specified conditions.

2. The sulfhydryl compound detection reagent according to claim 1, wherein the shield reagent is an aqueous solution of sodium iodate or an aqueous solution of sodium periodate.

3. The sulfhydryl compound detection reagent according to claim 2, wherein the shield reagent is the aqueous solution of sodium periodate.

4. The sulfhydryl compound detection reagent according to claim 1, wherein a preparation method of the PTA reagent is as follows: weighing 5±0.5 g of sodium tungstate, dissolving the sodium tungstate in 40±5 ml of deionized water, and adding 4±0.5 ml of 85±0.5% concentrated phosphoric acid and zeolite to obtain a resulting mixture; heating the resulting mixture to reflux, and continuously heating the resulting mixture under reflux for 2±0.5 h; and stopping heating, cooling to room temperature, diluting the resulting mixture to 100±0.5 ml to obtain a first resulting product, and storing the first resulting product in a first brown reagent bottle.

5. The sulfhydryl compound detection reagent according to claim 4, wherein
a preparation method of the acetate buffer is as follows: preparing a 2±0.5 M sodium acetate solution and a 2±0.5 M acetic acid solution separately, and mixing the 2±0.5 M sodium acetate solution and the 2±0.5 M acetic acid solution in a volume ratio of 5:1.

6. The sulfhydryl compound detection reagent according to claim 5, wherein a preparation method of the shield reagent is as follows: weighing 25 mg to 100 mg of a shield agent, dissolving the shield agent in 100±0.5 ml of deionized water to obtain a second resulting product, and storing the second resulting product in a second brown reagent bottle.

7. The sulfhydryl compound detection reagent according to claim 6, wherein the preparation method of the shield reagent is as follows: weighing 50 mg to 100 mg of the shield agent, dissolving the shield agent in 100±0.5 ml of deionized water to obtain the second resulting product, and storing the second resulting product in the second brown reagent bottle.

8. The sulfhydryl compound detection reagent according to claim 6, wherein the sulfhydryl compound detection reagent is obtained by thoroughly mixing the PTA reagent, the acetate buffer, and the shield reagent in a volume ratio of 2:1:1.

9. A preparation method of a sulfhydryl compound detection reagent, comprising the following steps:
S1: weighing 5±0.5 g of sodium tungstate, dissolving the sodium tungstate in 40±5 ml of deionized water, and adding 4±0.5 ml of 85±0.5% concentrated phosphoric acid and zeolite to obtain a resulting mixture; heating the resulting mixture to reflux, and continuously heating the resulting mixture under reflux for 2±0.5 h; and stopping heating, cooling to room temperature, diluting the resulting mixture to 100±0.5 ml to obtain a PTA reagent, and storing the PTA reagent in a first brown reagent bottle;
S2: preparing a 2±0.5 M sodium acetate solution and a 2±0.5 M acetic acid solution separately, and mixing the 2±0.5 M sodium acetate solution and the 2±0.5 M acetic acid solution in a volume ratio of 5:1 to obtain an acetate buffer;
S3: weighing 50 mg to 100 mg of a shield agent, dissolving the shield agent in 100±0.5 ml of deionized water to obtain a shield reagent, and storing the shield reagent in a second brown reagent bottle; and
S4: thoroughly mixing the PTA reagent, the acetate buffer, and the shield reagent in a volume ratio of 2:1:1.

10. A sulfhydryl compound detection reagent kit, comprising a sampler, a test reagent, and a test tube, wherein the test reagent is the sulfhydryl compound detection reagent according to claim 1.

11. A urine sulfhydryl compound detection test paper, wherein the urine sulfhydryl compound detection test paper is composed of a porous water-absorbent paper and a dry detection reagent dispersed on the porous water-absorbent paper, and the dry detection reagent is the sulfhydryl compound detection reagent according to claim 1.

12. A preparation method of a urine sulfhydryl compound detection test paper, comprising the following steps:
step 1: a preparation of a detection reagent comprising:
S1: weighing 5±0.5 g of sodium tungstate, dissolving the sodium tungstate in 40±5 ml of deionized water, and adding 4±0.5 ml of 85±0.5% concentrated phosphoric acid and zeolite to obtain a resulting mixture; heating the resulting mixture to reflux, and continuously heating the resulting mixture under reflux for 2±0.5 h; and stopping heating, cooling to room temperature, diluting the resulting mixture to 100±0.5 ml to obtain a PTA reagent, and storing the PTA reagent in a first brown reagent bottle;
S2: preparing a 2±0.5 M sodium acetate solution and a 2±0.5 M acetic acid solution separately, and mixing the 2±0.5 M sodium acetate solution and the 2±0.5 M acetic acid solution in a volume ratio of 5:1 to obtain an acetate buffer;
S3: weighing 50 mg to 100 mg of a shield agent, dissolving the shield agent in 100±0.5 ml of deionized water to obtain a shield reagent, and storing the shield reagent in a second brown reagent bottle; and
S4: thoroughly mixing the PTA reagent, the acetate buffer, and the shield reagent in a volume ratio of 2:1:0.5;
step 2: immersing a porous water-absorbent paper in the detection reagent; and after the porous water-absorbent paper fully absorbs the detection reagent, taking the porous water-absorbent paper out, and draining and drying the porous water-absorbent paper to obtain the urine sulfhydryl compound detection test paper.

13. The preparation method of the urine sulfhydryl compound detection test paper according to claim 12, wherein the porous water-absorbent paper is a filter paper; the porous water-absorbent paper is immersed at room temperature for no less than 5 seconds; and the porous water-absorbent paper is vacuum-dried at 40° C. to 60° C.

14. The preparation method of the urine sulfhydryl compound detection test paper according to claim 13, wherein when the porous water-absorbent paper is immersed, an ultrasonic-assisted treatment is conducted for no less than 10 seconds.

15. A sulfhydryl compound detection test paper kit, comprising a urine sampler, a detection test paper, and a desiccant, wherein the detection test paper is the urine sulfhydryl compound detection test paper according to claim 11.

16. The sulfhydryl compound detection reagent kit according to claim 10, wherein the shield reagent is an aqueous solution of sodium iodate or an aqueous solution of sodium periodate.

17. The sulfhydryl compound detection reagent kit according to claim 16, wherein the shield reagent is the aqueous solution of sodium periodate.

18. The sulfhydryl compound detection reagent kit according to claim 10, wherein a preparation method of the PTA reagent is as follows: weighing 5±0.5 g of sodium tungstate, dissolving the sodium tungstate in 40±5 ml of deionized water, and adding 4±0.5 ml of 85±0.5% concentrated phosphoric acid and zeolite to obtain a resulting mixture; heating the resulting mixture to reflux, and continuously heating the resulting mixture under reflux for 2±0.5 h; and stopping heating, cooling to room temperature, diluting the resulting mixture to 100±0.5 ml to obtain a first resulting product, and storing the first resulting product in a first brown reagent bottle.

19. The sulfhydryl compound detection reagent kit according to claim 18, wherein
a preparation method of the acetate buffer is as follows: preparing a 2±0.5 M sodium acetate solution and a 2±0.5 M acetic acid solution separately, and mixing the 2±0.5 M sodium acetate solution and the 2±0.5 M acetic acid solution in a volume ratio of 5:1.

20. The sulfhydryl compound detection reagent kit according to claim 19, wherein a preparation method of the shield reagent is as follows: weighing 25 mg to 100 mg of a shield agent, dissolving the shield agent in 100±0.5 ml of deionized water to obtain a second resulting product, and storing the second resulting product in a second brown reagent bottle.
